(19) United States
(12) Patent Application Publication
(10) Pub. No.: US 2012/0029388 A1
(43) Pub. Date: Feb. 2, 2012

(54) FORCE BEARING MONITOR APPARATUS FOR SPORTS SHOES

(76) Inventor: Cheng-Tang CHIANG, Nantou County (TW)

(21) Appl. No.: 12/846,192

(22) Filed: Jul. 29, 2010

(51) Int. Cl. A61B 5/103

(52) U.S. Cl. 600/592

(57) ABSTRACT

A force bearing monitor apparatus for sports shoes includes a first accelerometer module located at a rear end of a sole of a shoe and a second accelerometer module located at a front end of the sole to measure alterations of a first acceleration and a second acceleration borne by the shoe when a user is stridden and stepped during running. Through generation sequence of the alterations of the first and second accelerations, an exercise mode is determined. And through processing of a first signal processing unit and an interrelation processing unit, a first reacting force and a second reacting force are derived, and the ratio of the first and second reacting forces is obtained to serve as a control factor to adjust softness and hardness of the sole. According to the exercise mode, the softness and hardness of the sole can be made in response to the ground.

### FIELD OF THE INVENTION

The present invention relates to footwear having a sole changed in softness and hardness in response to external conditions and particularly to a force bearing monitor apparatus to generate a control factor to adjust softness and hardness of a sole.

### BACKGROUND OF THE INVENTION

Shoes provide protection for wearer's feet and knees. A shoe has a sole at the bottom to absorb impact to prevent a wearer's knee from directly receiving reacting forces from the ground to avoid injury. It is an important feature of the shoe. To allow a pair of shoes to be suited to different grounds and exercises in varying environments, U.S. Pat. No. 7,188,439 discloses a pair of shoes equipped with a cushion pad adjustable automatically. The cushion pad can detect deformation amounts of a sole to automatically change the softness and hardness thereof to meet requirements of varying grounds and exercises of different occasions.

The aforesaid prior art adjusts the softness and hardness of the cushion pad by simply detecting the deformation amounts of the cushion pad. It relies on a simplified control factor and often results in unsuitable change of the softness and hardness. For instance, if a wearer steps lightly, smaller deformation amounts take place. Thus a misinterpretation of exercising on a softer ground is made and the cushion pad is adjusted to make the sole harder. This could result in injury to the wearer's knee. On the other hand, if the wearer steps heavier, it could be misinterpreted exercising on a harder ground and the cushion pad is adjusted too soft. Hence it is difficult to control within a constant range and cannot fully meet actual requirements.

### SUMMARY OF THE INVENTION

The primary object of the present invention is to generate a control factor to adjust the softness and hardness of a sole within a constant range to meet requirements.

To achieve the foregoing object, the invention provides a force bearing monitor apparatus to generate a control factor to adjust softness and hardness of a sole. The apparatus comprises a first accelerometer module, a second accelerometer module, a first signal processing unit and an interrelation processing unit. The first accelerometer module is located at a rear end of a sole, and the second accelerometer module is located at a front end of the sole. The first and second accelerometer modules detect alterations of a first acceleration and a second acceleration when the shoe is stridden forwards and stepped. The first signal processing unit and interrelation processing unit process the alterations of the first and second accelerations and calculate a first reacting force and a second reacting force borne by the shoe, and their ratio also can be derived to generate the control factor, thereby to get an exercise mode.

In short, the invention provides many advantages, notably: by measuring the ratio of the first and second reacting forces to determine the control factor and obtain the exercise mode, and the influence of a user's artificial heavy or light footstep can be excluded to determine the control factor through the actual ground and exercise mode to fully meet requirements.

The foregoing, as well as additional objects, features and advantages of the invention will be more readily apparent from the following detailed description, which proceeds with reference to an embodiment and the accompanying drawings. The embodiment serves merely for illustrative purpose and is not the limitation of the invention.

### BRIEF DESCRIPTION OF THE DRAWINGS

FIG. 1 is a system block diagram of the invention.

FIG. 2A is a schematic view of measuring the first reacting force according to the invention.

FIG. 2B is a schematic view of measuring the second reacting force according to the invention.

FIG. 3 is a schematic view of an embodiment of the invention adopted on a shoe.

FIG. 4 is a system block diagram of the invention adopted on a shoe equipped with a control module.

FIG. 5 is a schematic view of the invention in a use condition.

### DETAILED DESCRIPTION OF THE PREFERRED EMBODIMENT

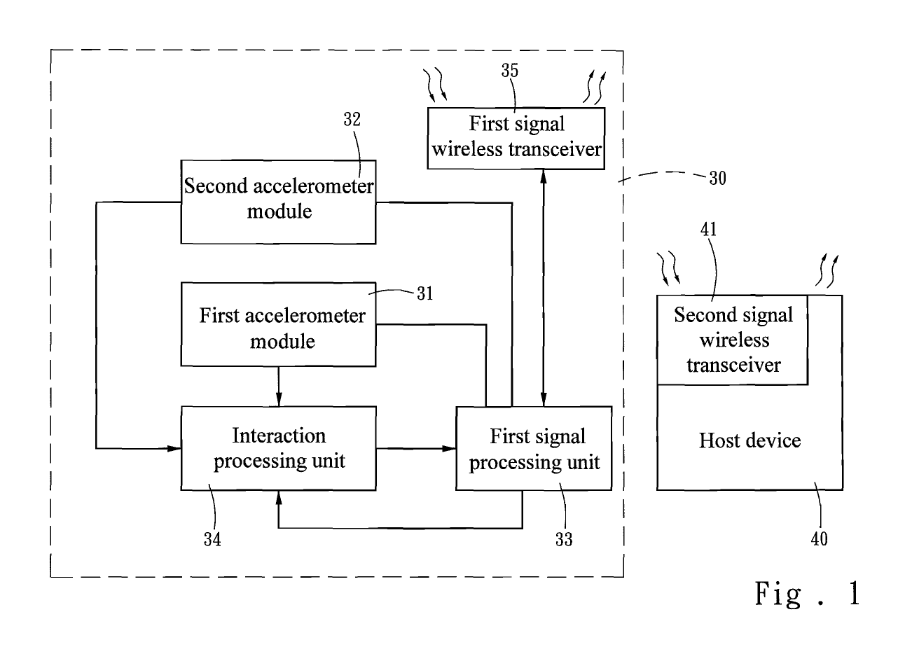

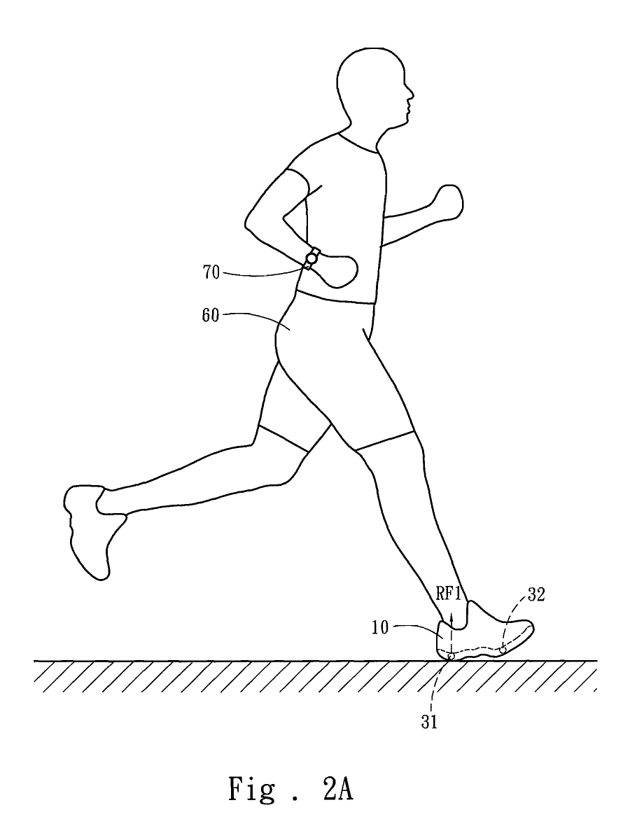

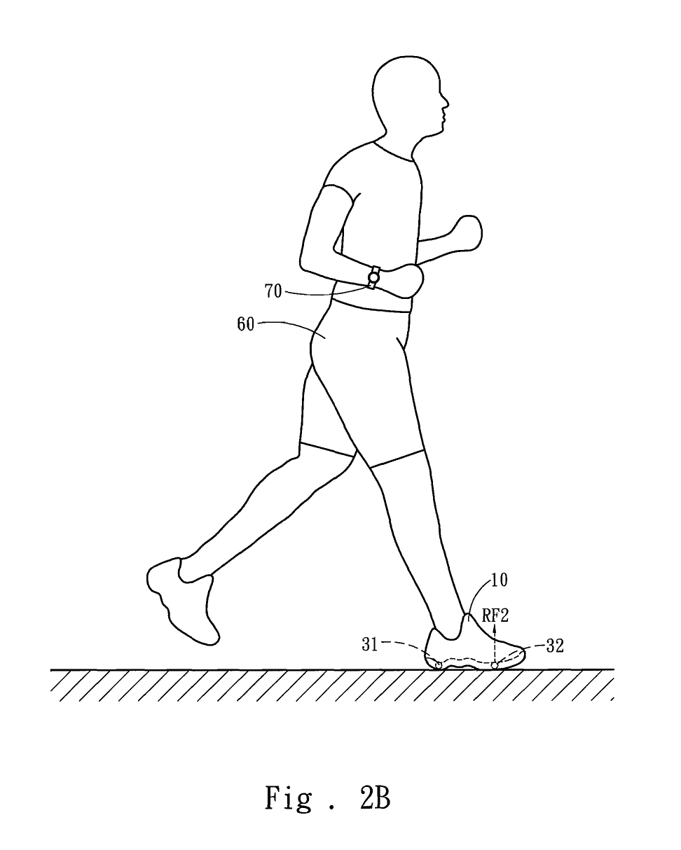

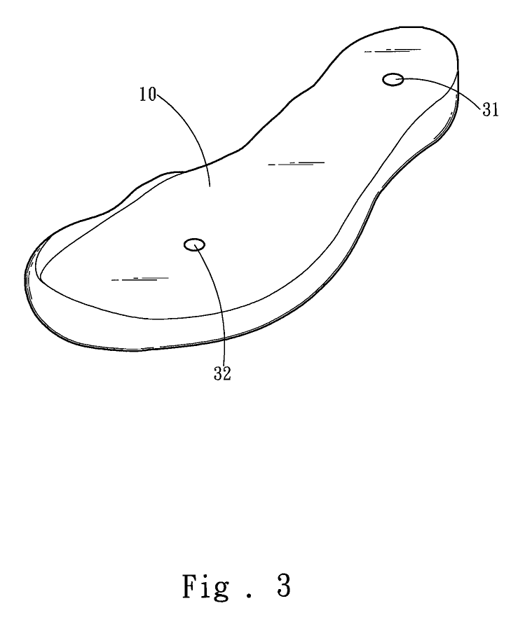

Referring to FIGS. 1, 2A, 2B, and 3, the invention includes a measuring device 30 which is linked wirelessly to a host device 40 equipped with a second signal wireless transceiver 41 through a first signal wireless transceiver 35. The measuring device 30 comprises a first accelerometer module 31, a second accelerometer module 32, a first signal processing unit 33 linked to the first signal wireless transceiver 35 and an interrelation processing unit 34. The first accelerometer module 31 is located at a rear end of the sole of a shoe 10 to measure alterations of a first acceleration borne by the rear heel when the shoe 10 is stridden and stepped (referring to FIG. 2A). The second accelerometer module 32 is located at a front end of the sole of the shoe 10 to measure alterations of a second acceleration borne by the front heel when the shoe 10 is stridden and stepped (referring to FIG. 2B). The first signal processing unit 33 and interrelation processing unit 34 are linked to the first accelerometer module 31 and second accelerometer module 32, and process the alterations of the first acceleration and second acceleration against time to calculate a first reacting force RF1 (referring to FIG. 2A) and a second reacting force RF2 (referring to FIG. 2B) borne by the shoe 10, and the ratio of the first reacting force RF1 and second reacting force RF2 is obtained to generate a control factor.

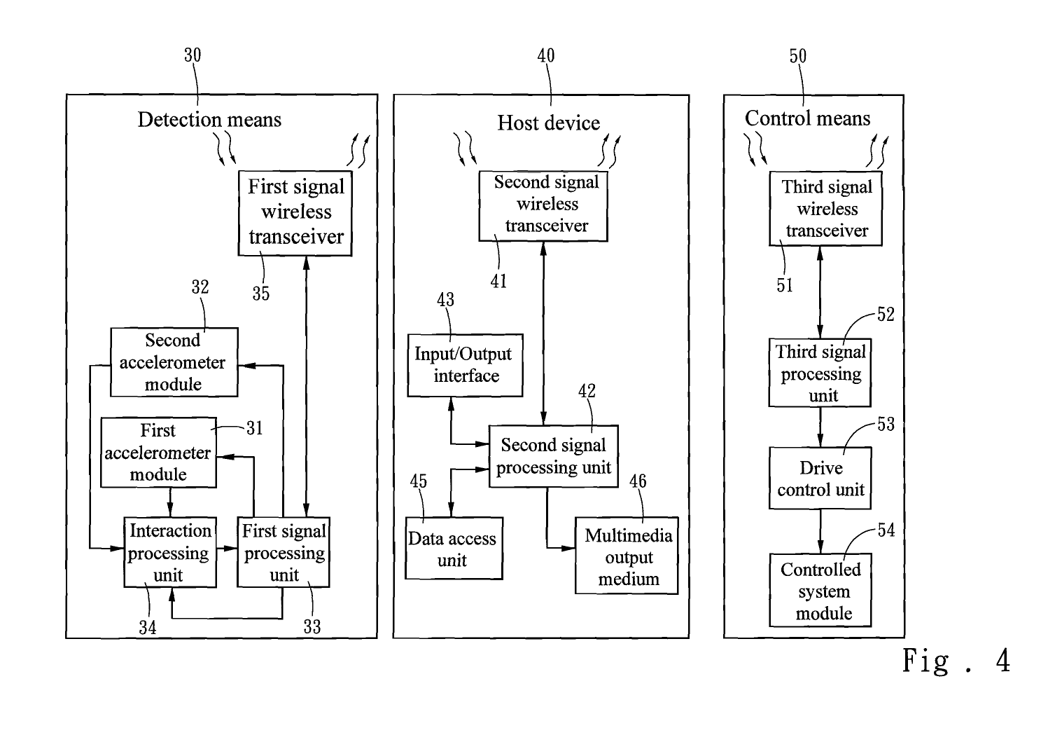

Referring to FIG. 4, the measuring device 30 of the invention aims to generate the control factor, and can be incorporated with a host device 40 and a control device 50 when in use. The host device 40 includes a second signal wireless transceiver 41, a second signal processing unit 42, an input/output interface 43, a data access unit 45 and a multimedia output medium 46. The control device 50 includes a third signal wireless transceiver 51, a third signal processing unit 52, a driving control unit 53 and a controlled system module 54. The measuring device 30, host device 40 and control device 50 are interconnected wirelessly through the first, second and third signal wireless transceivers 35, 41 and 51. The host device 40 is linked to the second signal wireless transceiver 41 through the second signal processing unit 42 to control the measuring device 30 and control device 50, linked to the input/output interface 43 to input/output a control parameter, connected to the data access unit 45 to access data, and linked to the multimedia output medium 46 to output multimedia information.

The control device 50 is linked to the third signal wireless transceiver 51 and driving control unit 53 through the third signal processing unit 52 to receive control signals from the host device 40, and linked to the controlled system module 54 through the driving control unit 53 to enable the controlled system module 54 to change the softness and hardness of the sole according to the control factor.

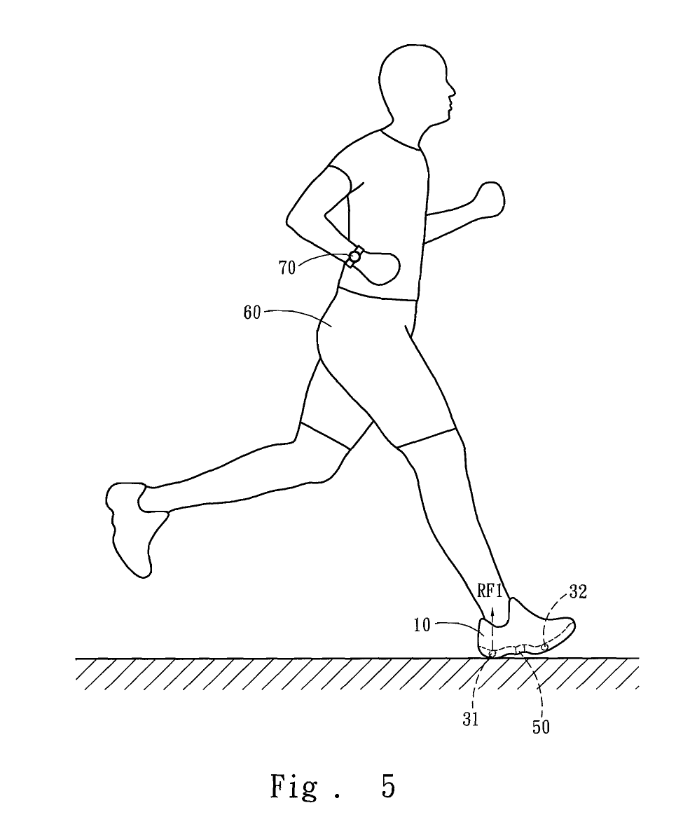

Refer to FIGS. 4 and 5 for an embodiment of the invention. The host device 40 can be installed on a wristwatch 70. The control device 50 is installed on a shoe 10. Through the controlled system module 54, the softness and hardness of the sole can be changed. When a user 60 wears the shoe 10, the wristwatch 70 and exercises, setting can be made through the input/output interface 43 of the host device 40 so that the controlled system module 54 can change the softness and hardness of the sole according to the control factor, namely the user 60 can set an exercise mode according to his/her own requirement to make the sole in a desired softness and hardness to meet his/her requirement. Thus, through the host device 40 on the wristwatch 70, the control factor of the measuring device 30 can be obtained, and the user 60 can instantly get the impact force (first reacting force RF1 and second reacting force RF2) borne by the shoe 10 through the wristwatch 70.

As a conclusion, the present invention determines the control factor through the ratio of the first and second reacting forces RF1 and RF2, hence can filter out artificial influence of heavy and light footsteps. Moreover, through the ratio of the first and second reacting forces RF1 and RF2 and generation sequence thereof, the user's exercise mode can be obtained, such as uphill or downhill movement. Thus the control factor of the invention can be used to determine the actual ground and exercise mode to adjust and control the softness and hardness of the sole to meet user's actual requirements.

What is claimed is:

1. A force bearing monitor apparatus for sports shoes, comprising a measuring device located on a sole of a shoe, the measuring device including:
a first accelerometer module located at a rear end of the sole of the shoe to measure alterations of a first acceleration borne by a user's rear heel when the shoe is stridden and stepped;
a second accelerometer module located at a front end of the sole of the shoe to measure alterations of a second acceleration borne by a user's front heel when the shoe is stridden and stepped;
a first signal processing unit linked to the first accelerometer module and the second accelerometer module; and
an interrelation processing unit linked to the first accelerometer module and the second accelerometer module; the first signal processing unit and the interrelation processing unit processing the alterations of the first acceleration and the second acceleration against time to get a first reacting force and a second reacting force borne by the shoe, and the ratio of the first reacting force and the second reacting force is obtained to generate a control factor.

2. The force bearing monitor apparatus of claim 1 further including a host device which comprises a second signal wireless transceiver, a second signal processing unit and an input/output interface; the measuring device including a first signal wireless transceiver, the measuring device being wirelessly linked to the host device through the first signal wireless transceiver and the second signal wireless transceiver, and the host device being linked to the input/output interface through the second signal processing unit to input/output a control parameter.

3. The force bearing monitor apparatus of claim 2, wherein the host device further includes a data access unit linked through the second signal processing unit to access data.

4. The force bearing monitor apparatus of claim 2, wherein the host device further includes a multimedia output medium linked through the second signal processing unit to output multimedia information.

5. The force bearing monitor apparatus of claim 2 further including a control device which comprises a third signal wireless transceiver, a third signal processing unit, a driving control unit and a controlled system module; the measuring device, host device and control device being interconnected wirelessly through the first, second and third signal wireless transceivers, the control device being linked to the third signal wireless transceiver and the driving control unit through the third signal processing unit to receive control signals from the host device and be connected to the controlled system module through the driving control unit.

6. The force bearing monitor apparatus of claim 5, wherein the host device is installed on a wristwatch and the control device is installed on the shoe.
